(19) United States
(12) Patent Application Publication
(10) Pub. No.: US 2015/0359816 A1
(43) Pub. Date: Dec. 17, 2015

(54) STABLE COMPOSITIONS COMPRISING HEPARINOID, ACUTE-ACTING ANESTHETIC, AND BUFFER

(71) Applicant: URIGEN PHARMACEUTICALS, INC., Wilmington, DE (US)

(72) Inventor: C. Lowell PARSONS, San Diego, CA (US)

(21) Appl. No.: 14/763,369

(22) PCT Filed: Jan. 28, 2014
(86) PCT No.: PCT/US14/13352

§ 371 Date: Jul. 24, 2015

(51) Int. Cl. A61K 31/727; A61K 31/167

(52) U.S. Cl. CPC A61K 31/727; A61K 31/167

(57) ABSTRACT

Improved methods for preparing compositions including a heparinoid, an acute-acting anesthetic, and a buffer are described. These methods result in compositions in which the heparinoid and the acute-acting anesthetic are at least 90% stable for one year. Compositions prepared by these methods and having such improved stability properties are also described, as well as methods for use of these compositions for treating, ameliorating, or preventing lower urinary tract disorders such as interstitial cystitis.

### CROSS-REFERENCES

This application claims the benefit of U.S. Provisional Patent Application Ser. No. 61/757,592 by C. L. Parsons, filed Jan. 28, 2013 and entitled “Stable Compositions Comprising Heparinoid, Acute-Acting Anesthetic, and Buffer,” the contents of which are incorporated herein in their entirety by this reference.

### FIELD OF THE INVENTION

This application is directed to stable compositions comprising a heparinoid, an acute-acting anesthetic, and a buffer, and methods for their preparation.

### BACKGROUND OF THE INVENTION

Interstitial cystitis (IC) is a chronic progressive disorder of the lower urinary tract that causes urinary urgency and frequency and/or pelvic pain. For many years, urologists regarded IC as a rare disease for which they had no broadly effective treatment. In fact, the condition is quite common. In 1999, prevalence in the United States was estimated at 750,000 cases (Curhan, et al. J Urol 161(2):549-552 (1999)). However the true prevalence of IC is estimated to be at least 1-2 million patients who are suffering from severe chronic pelvic pain. In addition overactive bladder, urethral syndrome, prostatitis, and gynecologic chronic pelvic pain syndrome comprises millions of patients that also result in bladder symptoms of urgency, frequency, incontinence and/or pelvic pain with no effective therapy and all these syndromes share a common pathophysiology with traditionally diagnosed IC (Parsons, C L Int Br J Urol December, 2010); there are no broadly effective treatments for these conditions.

Therefore, treatments that would both benefit a larger portion of the patient population, provide immediate relief of symptoms without causing additional pain, without requiring extensive alterations in diet, and further provide reversal of the disease process over time are necessary.

Compositions and methods for the treatment of interstitial cystitis are described in U.S. Pat. No. 7,414,039 to Parsons, issued Aug. 19, 2008 and entitled “Interstitial Therapy for Immediate Symptom Relief and Chronic Therapy in Interstitial Cystitis,” and in United States Patent Application Publication No. 2008/0300219 by Parsons, published Dec. 4, 2008 and entitled “Novel Interstitial Therapy for Immediate Symptom Relief and Chronic Therapy in Interstitial Cystitis,” both of which are incorporated herein in their entirety by this reference, as well as PCT Patent Application Publication No. WO 2006/07663 by Flashner et al., published Jul. 20, 2006 and entitled “Kits and Improved Compositions for Treating Lower Urinary Tract Disorders,” and PCT Patent Application Publication No. WO 2007/073397 by Flashner et al., published Jun. 28, 2007 and entitled Kits and Improved Compositions for Treating Lower Urinary Tract Disorders,” both of which are incorporated herein in their entirety by this reference. In general, the compositions disclosed in this issued patent and these published patent applications comprise an acute-acting anesthetic, typically lidocaine, a heparinoid, typically heparin, and a buffer, that are instilled directly into the urinary bladder.

Alkalinized lidocaine and heparin can be used to successfully treat bladder symptoms such as, but not limited to, urinary frequency, urgency, incontinence and bladder generated pain. Pain generated by the urinary bladder (a visceral organ) is not always perceived to be arising from the bladder. Pain can be referred anywhere from the navel to the knees and will also refer from the lumbar area down the buttocks to the legs and often has no relation to bladder filling or emptying. Consequently the origin of pelvic pain may not be recognized to be from the bladder. These bladder symptoms can be seen in a variety of “clinical syndromes” which may actually be all from one disease process: a dysfunctional epithelium (Parsons, C L Int Br J Urol, December 2010)). Nonetheless all these syndromes that can generate bladder symptoms that can be successfully treated with this solution, including, but not limited to, overactive bladder, interstitial cystitis, urethral syndrome in women, recurrent lower urinary tract infection, prostatitis (male chronic pelvic pain syndrome), radiation cystitis, chemical cystitis, gynecologic chronic pelvic pain syndrome (e.g. endometriosis, vulvodynia, vulvovaginitis, yeast vaginitis).

However, there is a problem in mixing these three compounds, as the wrong balance will result in the precipitation of lidocaine and loss of efficacy. Lidocaine when exposed to pH's at or above 7.0 will deionize and absorb through lipid membranes such as the bladder epithelium. As a result, the absorbed lidocaine will anesthetize the bladder nerves and relieve bladder symptoms noted above. The heparin will “coat” the bladder wall and inhibit the diffusion of potassium that is provoking the bladder symptoms in the first place. So the combination provides prolonged relief of bladder symptoms (Parsons, Urology 2003). However, mixing the heparin, lidocaine, and buffering agent has to be done in an exact way to prevent the precipitation of the lidocaine since lidocaine can precipitate at pH values above 7 depending on the conditions. The precipitation of lidocaine reduces its bioavailability and reduces the efficacy of the composition. Typically, the result will include the stabilizing of alkalinized (free base) lidocaine at from about 2% to about 45%.

For these reasons, there is a need for an improved process for compositions that include a heparinoid, an acute-acting anesthetic, and a buffer in order that these compositions can remain stable for a substantial period of time without precipitation or decomposition of the acute-acting anesthetic, as well as for stable compositions manufactured by such an improved process. There is a particular need for an improved process for manufacturing compositions that include heparin, lidocaine, and a physiologically compatible buffer, as well as for stable compositions that heparin, lidocaine, and bicarbonate buffer and that are manufactured by such as improved process. More specifically, there is a need for a method for manufacturing a solution that allows between about 2% and about 45% of the lidocaine to be present as the free base, which is the pharmaceutically active form, as well as a need for compositions in which between about 2% and about 45% of the lidocaine is present as the free base.

### SUMMARY OF THE INVENTION

An improved method of preparation of a composition including a heparinoid such as heparin, an acute-acting anesthetic such as lidocaine, and a buffer such as, prevents precipitation or decomposition of the acute-acting anesthetic such as lidocaine and thus maintains the bioavailability of the acute-acting anesthetic and the heparinoid. This maintains the stability and efficacy of the composition. Typically, this results in a composition in which, when the acute-acting anesthetic is lidocaine, between about 2% and about 45% of the lidocaine is present as the free base. It has unexpectedly been shown that heparinoids both stabilize the acute-acting anesthetic, such as lidocaine, in the composition, and promote absorption of the acute-acting anesthetic, such as lidocaine, by the urothelium.

One aspect of the invention is a method for preparing a composition useful for treatment of a lower urinary tract disease or condition comprising a heparinoid, an acute-acting anesthetic, and a buffer, the method comprising the steps of:

(1) providing a heparinoid, either as a solid or as an aqueous liquid, in a quantity of about 100 units to about 250,000 units per unit dose, or, alternatively, from about 0.5 mg to 1250 mg per unit dose;

(2) providing an acute-acting anesthetic, either as a solid or as an aqueous liquid, in a quantity of from about 5 mg to about 1000 mg per unit dose;

(3) combining the heparinoid and the acute-acting anesthetic; and

(4) buffering the combination of the heparinoid and the acute-acting anesthetic of step (3) to a pH value of greater than about 6.8 to about 8.3 with a buffer and the possible addition of a base selected from the group consisting of sodium hydroxide and potassium hydroxide compatible with both the heparinoid and the acute-acting anesthetic to form a stable solution.

Typically, the acute-acting anesthetic is lidocaine. Typically, when the resulting composition is intended for instillation into the bladder, and base is used in step (4), the base is sodium hydroxide, because the presence of potassium ions may aggravate certain urological conditions such as interstitial cystitis.

Another aspect of the invention is a method for preparing a composition useful for treatment of a lower urinary tract disease or condition comprising a heparinoid, an acute-acting anesthetic, and a buffer, the method comprising the steps of:

(1) providing a heparinoid, either as a solid or as an aqueous liquid, in a quantity of about 100 units to about 250,000 units per unit dose, or, alternatively, from about 0.5 mg to about 1250 mg per unit dose;

(2) buffering the heparinoid to a pH value of greater than about 6.8 to about 8.3 with a buffer compatible with both the heparinoid and an acute-acting anesthetic that is to be added subsequently;

(3) adding an acute-acting anesthetic, either as a solid or as an aqueous liquid, in a quantity of from about 5 mg to about 1000 mg per unit dose, to the buffered heparinoid from step (2) to form a solution including heparinoid, acute-acting anesthetic, and buffer; and

(4) if required, rebuffering the solution of step (3) to a pH value of greater than about 6.8 to about 8.3 to form a stable solution.

In these processes, the final pH is preferably from about 7.2 to about 7.6. More preferably, the final pH is about 7.3 to 7.5.

Typically, the heparinoid is selected from the group consisting of heparin, chondroitin sulfate, heparan sulfate, hyaluronic acid, keratan sulfate, dermatan sulfate, hyaluronan, and sodium pentosanpolysulfate. Preferably, the heparin is selected from the group consisting of heparin and sodium pentosanpolysulfate. More preferably, the heparinoid is heparin, such as heparin sodium.

Typically, the acute-acting anesthetic is selected from the group consisting of benzocaine, lidocaine, tetracaine, bupivacaine, cocaine, etidocaine, flecainide, mepivacaine, pramoxine, prilocaine, procaine, chloroprocaine, oxyprocaine, proparacaine, ropivacaine, dyclonine, dibucaine, propoxycaine, chloroxylenol, dexivacaine, diamocaine, hexylcaine, levobupivacaine, propoxycaine, pyrrocaine, risocaine, rodocaine, and pharmaceutically acceptable derivatives and bioisosteres thereof, and a combination thereof. Preferably, the acute-acting anesthetic is selected from the group consisting of lidocaine, bupivacaine, benzocaine, tetracaine, etidocaine, flecainide, prilocaine, and dibucaine, and a combination thereof. More preferably, the acute-acting anesthetic is lidocaine, such as lidocaine hydrochloride.

Typically, the buffer is selected from the group consisting of phosphate buffer, bicarbonate buffer, Tris (Tris(hydroxymethyl)aminomethane) buffer, MOPS buffer (3-(N-morpholino)propanesulfonic acid), HEPES (N-(2-hydroxyethyl)piperazine-N-(2-ethanesulfonic acid) buffer, ACES (2-[(2-amino-2-oxoethyl)amino]ethanesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid) buffer, HEPPSO (N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer, TAPS (N-tris[hydroxymethyl)methyl-3-aminopropanesulfonic acid) buffer; TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, 2-amino-2-methyl-1-propanol buffer, and a combination thereof. Preferably, the buffer is selected from the group consisting of bicarbonate buffer, Tris buffer, phosphate buffer, and a combination thereof.

The method can further comprise the step of adding one or more of: (i) an osmolar component; (ii) a compound that enables persistence of the composition to the surface of the bladder epithelium; (iii) an antibacterial agent; (iv) an antifungal agent; (v) a vasoconstrictor; or (vi) a preservative, subsequent to preparation of a buffered composition including the heparinoid, the acute-acting anesthetic, and the buffer.

In one alternative, both the heparinoid and the acute-acting anesthetic are provided in solid form; the solid form can be a powdered form. In another alternative, both the heparinoid and the acute-acting anesthetic are provided in liquid form. In yet another alternative, the heparinoid is provided in solid form and the acute-acting anesthetic is provided in liquid form; the solid form for the heparinoid can be a powdered form. In still another alternative, the heparinoid is provided in liquid form and the acute-acting anesthetic is provided in solid form; the solid form for the acute-acting anesthetic can be a powdered form. However, when the acute-acting anesthetic is lidocaine and the heparinoid is heparin, it is preferred to provide the lidocaine and heparin in powdered form.

Preferably, the heparinoid is heparin, the acute-acting anesthetic is lidocaine, and the buffer is a bicarbonate, tris or phosphate buffer. More preferably, the heparin is heparin sodium, the acute-acting anesthetic is lidocaine hydrochloride, and the buffer is phosphate buffer.

Typically, the quantity of heparin in the composition is from about 1000 units to about 250,000 units per unit dose of the composition, or, alternatively, from about 0.5 mg to about 1250 mg per unit dose of the composition. In various preferred alternatives, the quantity of heparin in the composition can be about 40,000 units, 50,000 units, or 60,000 units per unit dose of the composition, or, alternatively, about 200 mg, 250 mg, or 300 mg per unit dose of the composition. The conversion factor used herein is that 1 mg of heparin is approximately equivalent to 200 units of heparin.

Typically, the quantity of lidocaine in the composition is from about 10 mg to about 400 mg of lidocaine per unit dose. In a preferred alternative, the quantity of lidocaine in the composition can be about 200 mg of lidocaine per unit dose.

Yet another aspect of the present invention is a method for preparing a composition useful for treatment of a lower urinary tract disease or condition comprising a heparinoid, an acute-acting anesthetic, and a buffer, the method comprising the steps of:

(1) mixing the heparinoid and the acute-acting anesthetic to produce a liquid form in which the heparinoid and the acute-acting anesthetic are slightly more concentrated than in the final product;

(2) adding the buffer to produce a pH of about 7.0 to 7.3 in the solution of (1); and

(3) raising the pH to a value in the range of from about 7.1 to about 8.3 using sodium hydroxide and adding water as required to achieve the final desired concentrations of the heparinoid and the acute-acting anesthetic.

Another aspect of the composition is a stable composition comprising a heparinoid, an acute-acting anesthetic, and a buffer. Typically, the stability of the heparinoid and the acute-acting anesthetic is at least 90% after one year, up to 18 months. Preferably, the stability of the heparinoid and the acute-acting anesthetic is at least 95% after one year, up to 18 months. The composition can be prepared by a process as described above.

In such a composition according to the present invention, the heparinoid, the acute-acting anesthetic, and the buffer is as described above. Typical or preferred quantities of the heparinoid, the acute-acting anesthetic, and the buffer are as described above.

The composition can be formulated for treating, ameliorating, or preventing a lower urinary tract disorder selected from the group consisting of bacterial cystitis, fungal/yeast cystitis, vulvar vestibulitis, vulvodynia, dyspareunia, urethral syndrome, and endometriosis in women; prostatitis and chronic pelvic pain syndrome in men; and radiation-induced cystitis, chemotherapy-induced cystitis, interstitial cystitis, and overactive bladder in men or women. In particular, the composition can be formulated for treating, ameliorating, or preventing interstitial cystitis.

Yet another aspect of the invention is a method for treating, ameliorating, or preventing a lower urinary tract disorder comprising instillation of a therapeutically effective quantity of a composition according to the present invention into the bladder of a subject in need thereof, wherein the lower urinary tract disorder is selected from the group consisting of bacterial cystitis, fungal/yeast cystitis, vulvar vestibulitis, vulvodynia, dyspareunia, urethral syndrome, and endometriosis in women; prostatitis and chronic pelvic pain syndrome in men; and radiation-induced cystitis, chemotherapy-induced cystitis, interstitial cystitis, and overactive bladder in men or women. A particularly significant lower urinary tract disorder suitable for treatment by use of a composition according to the present invention is interstitial cystitis.

### DETAILED DESCRIPTION OF THE INVENTION

An improved method of preparation of a stable composition including a heparinoid such as heparin, an acute-acting anesthetic such as lidocaine, and a buffer, the following steps, in a first alternative:

(1) providing a heparinoid, either as a solid or as an aqueous liquid, in a quantity of about 100 units to about 250,000 units per unit dose, or, alternatively, from about 0.5 mg to about 1250 mg per unit dose;

(2) providing an acute-acting anesthetic, either as a solid or as an aqueous liquid, in a quantity of from about 5 mg to about 1000 mg per unit dose;

(3) combining the heparinoid and the acute-acting anesthetic; and

(4) buffering the combination of the heparinoid and the acute-acting anesthetic of step (3) to a pH value of greater than about 6.8 to about 8.3 with a buffer and the possible addition of a base selected from the group consisting of sodium hydroxide and potassium hydroxide compatible with both the heparinoid and the acute-acting anesthetic to form a stable solution.

Typically, the acute-acting anesthetic is lidocaine. Typically, when the resulting composition is intended for instillation into the bladder, and base is used in step (4), the base is sodium hydroxide, because the presence of potassium ions may aggravate certain urological conditions such as interstitial cystitis.

In a second alternative, the process comprises:

(1) providing a heparinoid, either as a solid or as an aqueous liquid, in a quantity of about 100 units to about 250,000 units per unit dose, or, alternatively, from about 0.5 mg to about 1250 mg per unit dose;

(2) buffering the heparinoid to a pH value of greater than about 6.8 to about 8.3 with a buffer compatible with both the heparinoid and an acute-acting anesthetic that is to be added subsequently;

(3) adding an acute-acting anesthetic, either as a solid or as an aqueous liquid, in a quantity of from about 5 mg to about 1000 mg per unit dose, to the buffered heparinoid from step (2) to form a solution including heparinoid, acute-acting anesthetic, and buffer; and

(4) if required, rebuffering the solution of step (3) to a pH value of greater than about 6.8 to about 8.3 to form a stable solution.

In these processes, the final pH is preferably from about 7.2 to about 7.6. More preferably, the final pH is about 7.3 to 7.5.

The results reported herein are quite surprising because alkalinized (free base) lidocaine can precipitate, and, as well, over time, the lidocaine can decompose. Use of methods according to the present invention and the resulting compositions allow the manufacture to mix all three components at the manufacturing stage and then sell or otherwise distribute a vial or other suitable dosage form that is premixed and only requires placement into the bladder, such as by instillation. The heparinoid prevents precipitation of the alkalinized lidocaine at high pH resulting in the presence of about 2% to about 45% lidocaine as the free base. This is simpler for both the doctor and the patient and reduces the risk of error or contamination of the solution to be instilled. Moreover, it has unexpectedly been shown that heparinoids both stabilize the acute-acting anesthetic, such as lidocaine, in the composition, and promote absorption of the acute-acting anesthetic, such as lidocaine, by the urothelium. Typically, it is preferred to premix the heparin and lidocaine from liquids or powders that have only sodium heparin and only lidocaine hydrochloride with no other agents so that in final buffering is does not result in lidocaine (or its equivalents) from precipitating. This involves mixing the two compounds and then adding buffer (whichever buffer is used, typically phosphate, Tris, or bicarbonate) to a pH of about 6.9 to 7.1 and then do the final pH adjustment with NaOH to a pH of 7.2 to 8.3. This gives the best lidocaine stability. If one use only buffer to raise the pH then lidocaine is less stable. It is a critical issue to have a stable solution.

Suitable and preferred alternatives for the heparinoid, the acute-acting anesthetic, and the buffer are described below. In one preferred alternative, the heparinoid is heparin, and the acute-acting anesthetic is lidocaine. In one particularly preferred alternative, the heparinoid is heparin sodium, the acute-acting anesthetic is lidocaine, and the buffer is sodium bicarbonate buffer, Tris buffer or phosphate buffer.

Solutions prepared by these processes show stability for the acute-acting anesthetic, such as lidocaine, and for the heparinoid, such as heparin of over 90% after one year, up to 18 months. Typically, solutions prepared by these processes show stability for the acute-acting anesthetic and the heparinoid of over 95% after one year, up to 18 months. Preferably, solutions prepared by these processes show stability for the acute-acting anesthetic and the heparinoid of over 97% after one year, up to 18 months.

A preservative can be added to the final stable solution including a heparinoid, an acute-acting anesthetic, and a buffer; this does not affect the stability.

As detailed below, when the heparinoid is heparin and the acute-acting anesthetic is lidocaine hydrochloride, it is necessary to employ powdered heparin and powdered lidocaine hydrochloride in the alternative processes described above, because available heparin and lidocaine hydrochloride solutions, such as USP Heparin and USP Lidocaine Hydrochloride, are not compatible upon the addition of buffer and the lidocaine may precipitate regardless of subsequent attempts to avoid precipitation and maintain the lidocaine in solution.

The composition can further comprise an osmolar component as described further below.

As used herein, “heparinoid” refers to any molecule comprising a glycosaminoglycan which refers to a molecule comprising a network of long, branched chains of sugars (e.g., chondroitin sulfate, heparan sulfate, hyaluronic acid, keratan sulfate, dermatan sulfate, hyaluronan, sodium pentosanpolysulfate, and the like) and optimally further comprising smaller, nitrogen-containing molecules (e.g. low molecular weight molecules). It is not meant to limit the present invention to any one glycosaminoglycan (GAG) or source of GAG. GAG molecules include but are not limited to low molecular weight (LMW) GAGs, naturally derived GAGs, biotechnologically prepared GAGs, chemically modified GAGs, synthetic GAGs, and the like. Heparinoids can also be comprised of pentoses instead of hexoses (GAGs are comprised of hexoses) such as pentosanpolysulfate. It is not meant to limit the present invention to any one heparinoid molecule or source of heparinoid molecule. As used herein, “heparin” refers to a heterogeneous group of straight-chain anionic glycosaminoglycans, as described above, with a molecular weight ranging from 2,000 to 40,000 Da. In some embodiments, heparin is a higher molecular weight species ranging from 8,000-40,000 daltons. As used herein, “low-molecular-weight heparins” refers to a lower molecular weight (LMW) species ranging from 2,000 to 8,000 daltons. Sodium pentosanpolysulfate can range from 2,000 to 6,000 daltons. Also included within the scope of the invention are polymers such as dalteparin or enoxaparin. LMW heparins are made by enzymatic or chemical controlled hydrolysis of unfractionated heparin and have very similar chemical structure as unfractionated heparin except for some changes that may have been introduced due to the enzymatic or chemical treatment. While not intending to limit the mechanism of action of the invention's compositions, the mechanism of action of these drugs may be similar to that of full-length heparin. LMW heparins are usually isolated from bulk heparin. In one embodiment, heparin or another heparinoid is a heparin salt. As used herein, the phrases “pharmaceutically acceptable salts”, “a pharmaceutically acceptable salt thereof” or “pharmaceutically accepted complex” for the purposes of this application are equivalent and refer to derivatives prepared from pharmaceutically acceptable non-toxic acids or bases including inorganic acids and bases and organic acids and bases.

Because of the negative charges of these polysaccharides due to the occurrence of sulfate groups and/or carboxylic acid groups in them, they are administered in the form of salts, with an appropriate cation to neutralize the negative charges on the acid groups. Typically, the cation is sodium. However, other physiologically tolerable counterions that do not induce urinary tract dysfunctions, such as magnesium, aluminum, calcium, ammonium, or salts made from physiologically acceptable organic bases such as, but not limited to, trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, dibenzylpiperidine, N-benzyl-p-phenethylamine, dehydroabietylamine, N,N′-bisdehydroabietylamine, glucamine, N-methylglucamine, collidine, quinine, quinoline, and basic amino acids such as lysine and arginine, can be used. These cationic counterions can alternatively be used as the counterions with anionic buffers such as bicarbonate, as well. Sodium is typically employed as the positively-charged counterion as indicated above; accordingly, a preferred form of heparin is heparin sodium in which sodium acts as the counterion. These salts may be prepared by methods known to those skilled in the art. However, it is generally undesirable to use potassium as a counterion due to its role in the etiology of the conditions and syndromes being treated. Other polysaccharides that have the required activity include, but are not limited, to dextran sulfate and carrageenan. Other glycosaminoglycans can be used in methods according to the invention, including low molecular weight (LMW) glycosaminoglycans, naturally derived glycosaminoglycans, biotechnologically prepared glycosaminoglycans, chemically modified glycosaminoglycans, and synthetic glycosaminoglycans and linear anionic polysaccharides comprised of pentoses. Reference to a heparinoid that possesses a negative charge at physiological pH, such as heparin, without specific reference to a counterion, is to be understood as including all possible counterions that do not interfere with the physiological activity of the heparin or other components of the composition and do not create incompatibility with any other components of the composition.

In some embodiments, a heparinoid comprises a heparin-like molecule (e.g. heparan sulfate). For example, a heparin-like molecule such as heparan sulfate is a glycocosaminoglycans with a structure similar to heparin with the difference being that heparan sulfate has undergone less polymerization than heparin and so has more glucuronic acid and N-acetyl glucosamine than heparin. Heparan sulfate contains fewer sulfate groups, so. Heparin exists in a variety of forms characterized by different degrees of sulfation. Typically, heparin has a molecular weight of from about 2 kDa to about 40 kDa. Heparin and heparan sulfate are both characterized by repeating units of disaccharides containing a uronic acid (glucuronic or iduronic acid) and glucosamine, which is either N-sulfated or N-acetylated. The sugar residues may be further 0-sulfated at the C-6 and C-3 positions of the glucosamine and the C-2 position of the uronic acid. There are at least 32 potential unique disaccharide units in this class of compounds. Five examples of sugars occurring in heparin are: (1) α-L-iduronic acid 2-sulfate; (2) 2-deoxy-2-sulfamino-α-D-glucose 6-sulfate; (3) β-D-glucuronic acid; (4) 2-acetamido-2-deoxy-α-D-glucose; and (5) α-L-iduronic acid.

In one embodiment, heparin contains at least 130 USP units per mg. Heparin is measured by its specific anticoagulation activity in units; either USP units or international units (IU) are specified in stating the activity of heparin. As used herein, “USP unit” refers to the quantity of heparin that prevents 1.0 ml of citrated sheep plasma from clotting for 1 hour after the addition of 0.2 ml of 1% CaCl₂ at 20° C. when compared to a USP reference standard (defined as units/nil). As used herein, “IU” refers to the quantity of heparin that is active in assays as established by the Fifth International standard for Unfractionated Heparin (WHO-5) (defined as International Units/nil) (Linhardt, R. J. & Gunay, N. S. (1999) Semin Thromb Hemost 25, 5-16.). However, it is also possible, and preferred in some embodiments, to specify the heparin concentration in terms of milligrams: typically, 1 mg of heparin is approximately equivalent to 200 units.

Particularly preferred heparinoids for use in methods according to the present invention and compositions prepared by those methods include heparin and sodium pentosanpolysulfate. A most particularly preferred heparinoid for use in methods according to the present invention and compositions prepared by those methods is heparin. A preferred form of heparin is heparin sodium, although, as described above, other counterions can be used. The quantity of heparin in compositions prepared according to methods of the present invention can range from about 1000 units to about 250,000 units per unit dose of the composition; any intermediate quantity of heparin, such as, but not limited to, 1,000 units, 5,000 units, 10,000 units, 15,000 units, 20,000 units, 25,000 units, 30,000 units, 35,000 units, 40,000 units, 45,000 units, 50,000 units, 55,000 units, 60,000 units, 65,000 units, 70,000 units, 75,000 units, 80,000 units, 85,000 units, 90,000 units, 95,000 units, 100,000 units, 110,000 units, 120,000 units, 130,000 units, 140,000 units, 150,000 units, 160,000 units, 170,000 units, 180,000 units, 190,000 units, 200,000 units, 210,000 units, 220,000 units, 230,000 units, 240,000 units, or 250,000 units per unit dose of the composition can be used. As expressed in milligrams, these quantities of heparin range from about 0.5 mg to about 1250 mg per unit dose, including but not limited to 5 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, or 1250 mg. Suitable quantities of heparinoids other than heparin can be determined by one of ordinary skill in the art based on the molecular weight of the heparinoid to be used.

The quantity of heparinoid in the composition can vary depending on the subject, the severity and course of the disease, the subject's health, the response to treatment, pharmacokinetic considerations such as liver and kidney function, and the judgment of the treating physician. Accordingly, a number of compositions including differing quantities of heparin per unit dose can be prepared by methods according to the present invention.

In accordance with the practice of the invention, merely by way of example, when the heparinoid is sodium pentosanpolysulfate, the amount of heparinoid in the composition may be about 1 mg to about 600 mg of sodium pentosanpolysulfate per unit dose (for example about 100 mg to about 600 mg per unit dose of sodium pentosanpolysulfate). In accordance with the practice of the invention, merely by way of example, when the heparinoid is heparan sulfate, the amount of heparinoid in the composition may be about 0.5 mg to about 10,000 mg of heparan sulfate per unit dose (for example about 100 mg to about 300 mg per unit dose of heparan sulfate). In accordance with the practice of the invention, merely by way of example, when the heparinoid is hyaluronic acid, the amount of heparinoid in the composition may be about 5 mg to about 600 mg of hyaluronic acid per unit dose (for example about 10 mg to about 100 mg per unit dose of hyaluronic acid). In accordance with the practice of the invention, merely by way of example, when the heparinoid is chondroitin sulfate, the amount of heparinoid in the composition may be about 1 mg to about 10,000 mg of chondroitin sulfate per unit dose (for example about 100 mg to about 300 mg per unit dose of chondroitin sulfate). In accordance with the practice of the invention, merely by way of example, when the heparinoid is heparin sodium, the amount of heparinoid in the composition may be about 10 mg to about 1000 mg of heparin sodium per unit dose.

The acute-acting anesthetic is typically a sodium channel blocker, such as, but not limited to, the drugs referred to commonly as the “caine” drugs, as well as other sodium channel blockers. The acute-acting anesthetic in a composition prepared according to the methods of the present invention can be, but is not limited to, any of benzocaine, lidocaine, tetracaine, bupivacaine, cocaine, etidocaine, flecainide, mepivacaine, pramoxine, prilocaine, procaine, chloroprocaine, oxyprocaine, proparacaine, ropivacaine, dyclonine, dibucaine, propoxycaine, chloroxylenol, dexivacaine, diamocaine, hexylcaine, levobupivacaine, propoxycaine, pyrrocaine, risocaine, rodocaine, and pharmaceutically acceptable derivatives and bioisosteres thereof, or a combination thereof. Preferably, the anesthetic (e.g., acute-acting anesthetic) is selected from the group consisting of lidocaine, bupivacaine, benzocaine, tetracaine, etidocaine, flecainide, prilocaine, and dibucaine, or a combination thereof. A particularly preferred acute-acting anesthetic is lidocaine; preferably, the lidocaine is in the form of lidocaine hydrochloride, in which the chloride acts as a counterion. As used herein, the recitation of an acute-acting anesthetic includes all salts of that acute-acting anesthetic that are compatible with the desired pH, the buffer used, and any counterions present; the recitation of an acute-acting anesthetic is not intended to limit the salt form or counterion used beyond these criteria. Specifically, reference to an acute-acting anesthetic that possesses a positive charge at physiological or near-physiological pH, such as lidocaine, without specific reference to a counterion, is to be understood as including all possible counterions that do not interfere with the physiological activity of the lidocaine or other components of the composition and do not create incompatibility with any other components of the composition.

The quantity of acute-acting anesthetic in the composition will vary depending on the subject, the severity and course of the disease, the subject's health, the response to treatment, pharmacokinetic considerations such as liver and kidney function, and the judgment of the treating physician. Accordingly, a number of compositions including differing quantities of acute-acting anesthetic per unit dose can be prepared by methods according to the present invention. For example, when the acute-acting anesthetic is lidocaine, such as lidocaine hydrochloride, the amount of lidocaine in the composition may be in the range of about 10 mg to about 400 mg per unit dose, any intermediate quantity of lidocaine, such as 10 mg, 20 mg, 30 mg, 40 mg. 50 mg, 60 mg, 70 mg, 80 mg, 90 mg, 100 mg, 110 mg, 120 mg. 130 mg, 140 mg, 150 mg, 160 mg, 170 mg, 180 mg, 190 mg, 200 mg, 220 mg, 240 mg, 260 mg, 280 mg, 300 mg, 320 mg, 340 mg, 360 mg, 380 mg, or 400 mg per unit dose of the composition can be used. For example, the amount of lidocaine can be 10 mL of 1% lidocaine per unit dose or 16 mL of 2% lidocaine per unit dose. In one preferred embodiment, the composition comprises 200 mg of lidocaine as lidocaine hydrochloride. Suitable quantities of acute-acting anesthetics other than lidocaine can be determined by one of ordinary skill in the art based on the molecular weight and anesthetic potency of the acute-acting anesthetic to be used.

The buffer in a composition prepared according to the methods of the present invention can be, but is not limited to, phosphate buffer, bicarbonate buffer, Tris (Tris(hydroxymethyl)aminomethane) buffer, MOPS buffer (3-(N-morpholino)propanesulfonic acid), HEPES (N-(2-hydroxyethyl)piperazine-N-(2-ethanesulfonic acid) buffer, ACES (2-[(2-amino-2-oxoethyl)amino]ethanesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid) buffer, HEPPSO (N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer, TAPS (N-tris[hydroxymethyl)methyl-3-aminopropanesulfonic acid) buffer; TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and 2-amino-2-methyl-1-propanol buffer, or a combination thereof. Particularly preferred buffers are bicarbonate buffer, phosphate buffer, Tris buffer or a combination thereof.

Because phosphate can bind up to three hydrogen ions, it can exist in several forms, including dihydrogen phosphate (H₂PO₄⁻), the monohydrogen phosphate (HPO₄²⁻), and the phosphate ion itself (PO₄³⁻). The pKₐ of the first ionization of phosphoric acid (H₃PO₄) to produce dihydrogen phosphate is about 2.12. The pKₐ of the ionization of dihydrogen phosphate to produce monohydrogen phosphate is about 7.21. The pKₐ of the ionization of monohydrogen phosphate to produce phosphate ion is about 12.67. The relative proportions of dihydrogen phosphate, monohydrogen phosphate, and phosphate ion present at a specified pH can readily be determined by use of the Henderson-Hasselbalch equation. Typically, when phosphate buffer is employed, it is employed as dihydrogen phosphate in view of the pH ranges involved; however, it is also possible to employ monohydrogen phosphate and add an alkalinizing agent such as sodium hydroxide to raise the pH to the desired value. Alternatively, a combination of monohydrogen phosphate and dihydrogen phosphate can be employed. Although it is possible to use other hydroxides such as potassium hydroxide, it is generally preferred to use sodium hydroxide in preference to potassium hydroxide in view of the potential role of potassium ion in the etiology of a number of lower urinary tract conditions. Phosphate buffer is a preferred buffer in some alternatives because it is more physiologically acceptable to the bladder and is normally present in urine.

In general, it is preferred to use an alkalinizing agent such as sodium hydroxide to achieve the final pH, rather than the buffer itself. The use of the alkalinizing agent to achieve the final pH results in greater stability of the acute-acting anesthetic, particularly lidocaine.

In one particularly preferred method of preparing a composition according to the present invention, the composition is prepared by the following process:

(1) mixing the heparinoid and the acute-acting anesthetic to produce a liquid form in which the heparinoid and the acute-acting anesthetic are slightly more concentrated than in the final product;

(2) adding the buffer to produce a pH of about 7.0 to 7.3 in the solution of (1); and

(3) raising the pH to a value in the range of from about 7.1 to about 8.3 using sodium hydroxide and adding water as required to achieve the final desired concentrations of the heparinoid and the acute-acting anesthetic.

Typically, the pH value obtained in step (3) is about 7.3 to 7.5. Typically, in a composition prepared according to the above-identified three-step process, the heparinoid is heparin, the buffer is sodium bicarbonate, Tris or sodium phosphate, and the acute-acting anesthetic is lidocaine. In another alternative of a composition prepared according to the above-identified three-step process, the heparinoid is heparin, the buffer is phosphate buffer, and the acute-acting anesthetic is lidocaine.

The quantity of buffer in the composition will vary depending on the subject, the severity and course of the disease, the subject's health, the response to treatment, pharmacokinetic considerations such as liver and kidney function, and the judgment of the treating physician. Accordingly, a number of compositions including differing quantities of buffer per unit dose can be prepared by methods according to the present invention. For example, when the buffer is sodium bicarbonate, the amount of sodium bicarbonate may be about 3 mL of 8.4% sodium bicarbonate per unit dose.

A particularly preferred composition prepared by methods according to the present invention can comprise heparin sodium as the heparinoid, lidocaine hydrochloride as the acute-acting anesthetic, and sodium bicarbonate, Tris or sodium phosphate as the buffer.

Compositions prepared by methods according to the present invention can comprise one or more additional optional components. Such additional optional components can include:

(1) an osmolar component that provides an isotonic or nearly isotonic solution compatible with human cells and blood;

(2) a compound that enables persistence of the composition to the surface of the bladder epithelium in a quantity sufficient to treat, ameliorate, or prevent a lower urinary tract disorder;

(3) an antibacterial agent in a quantity sufficient to treat, ameliorate, or prevent a lower urinary tract disorder;

(4) an antifungal agent in a quantity sufficient to treat, ameliorate, or prevent a lower urinary tract disorder;

(5) a vasoconstrictor in a quantity sufficient to treat, ameliorate, or prevent a lower urinary tract disorder;

(6) a preservative; and

(7) an anti-inflammatory agent.

When present, the optional osmolar component is a salt, such as sodium chloride, or a sugar or a combination of two or more of these components. The sugar may be a monosaccharide such as dextrose, a disaccharide such as sucrose or lactose, a polysaccharide such as dextran 40, dextran 60, or starch, or a sugar alcohol such as mannitol. It should be obvious to those skilled in the art that all components of the solution contribute to the osmolarity of the solution but to achieve an isotonic or near-isotonic solution, the contributions of those components should be taken into account to ensure that the proper proportion of osmolar component is added and an excess of osmolar component is not added which would result in a hypertonic solution. In fact, when the composition described above including heparin sodium as the heparinoid, lidocaine hydrochloride as the anesthetic, and sodium bicarbonate as the buffer, the osmolar contributions of the sodium ion from the heparin sodium and sodium bicarbonate, the chloride ion from the lidocaine hydrochloride, and the carbonate/bicarbonate ion from the sodium bicarbonate are sufficient not to require an additional osmolar component.

If an antibacterial agent is present, the antibacterial agent can be selected from the group consisting of a sulfonamide, a penicillin, a combination of trimethoprim plus sulfamethoxazole, a quinolone, methenamine, nitrofurantoin, a cephalosporin, a carbapenem, an aminoglycoside, a tetracycline, and a macrolide. Suitable sulfonamides include, but are not limited to, sulfanilamide, sulfadiazine, sulfamethoxazole, sulfisoxazole, sulfamethizole, sulfadoxine, and sulfacetamide. Suitable penicillins include, but are not limited to, methicillin, nafcillin, oxacillin, cloxacillin, dicloxacillin, ampicillin, amoxicillin, bacampicillin, carbenicillin, ticarcillin, mezlocillin, and piperacillin. Suitable quinolones include, but are not limited to, nalidixic acid, cinoxacin, norfloxacin, ciprofloxacin, orfloxacin, sparfloxacin, lomefloxacin, fleroxacin, pefloxacin, and amifloxacin. Suitable cephalosporins include, but are not limited to, cephalothin, cephazolin, cephalexin, cefadroxil, cefamandole, cefoxatin, cefaclor, cefuroxime, loracarbef, cefonicid, cefotetan; ceforanide, cefotaxime, cefpodoxime proxetil, ceftizoxime, ceftriaxone, cefoperazone, ceftazidime, and cefepime. Suitable carbepenems include, but are not limited to, imipenem, meropenem, and aztreonam. Suitable aminoglycosides include, but are not limited to, netilmycin and gentamicin. Suitable tetracyclines include, but are not limited to, tetracycline, oxytetracycline, demeclocycline, minocycline, doxycycline, and chlortetracycline. Suitable macrolides include, but are not limited to, erythromycin, clarithromycin, and azithromycin.

If an antifungal agent is present, the antifungal agent can be selected from the group consisting of amphotericin B, itraconazole, ketoconazole, fluconazole, miconazole, and flucytosine.

If a vasoconstrictor is present, the vasoconstrictor can be epinephrine.

If a compound that enables persistence of the composition to the surface of the bladder epithelium is present, the compound is typically an activatable gelling agent. The activatable gelling agent is typically a thermoreversible gelling agent. The thermoreversible gelling agent can be selected from the group consisting of Pluronics F127 gel, Lutrol gel, N-isopropylacrylamide, ethylmethacrylate, N-acryloxysuccinimide, xyloglucan sols of 1-2%, graft copolymers of pluronic and poly(acrylic acid), pluronic-chitosan hydrogels, and a [poly(ethylene glycol)-poly[lactic acid-co-glycolic acid]-poly(ethylene glycol)] (PEG-PLGA-PEG) copolymer.

If a preservative is present, the preservative can be selected from the group consisting of parabens, chlorobutanol, phenol, sorbic acid, or thimerosal. However, typically, compositions prepared by methods according to the present invention do not require a preservative component, as the compositions are prepared and dispensed in sealed single-unit-dose vials.

If an anti-inflammatory agent is present, the anti-inflammatory agent can be a steroid or a non-steroidal anti-inflammatory agent. Suitable steroids and non-steroidal anti-inflammatory agents are known in the art. Suitable steroids include, but are not limited to, hydrocortisone, cortisone, beclomethasone dipropionate, betamethasone, dexamethasone, prednisone, methylprednisolone, triamcinolone, fluocinolone acetonide, and fludrocortisone. Suitable non-steroidal anti-inflammatory agents include, but are not limited to, acetylsalicylic acid (aspirin), sodium salicylate, choline magnesium trisalicylate, salsalate, diflunisal, sulfasalazine, olsalazine, acetaminophen, indomethacin, sulindac, tolmetin, diclofenac, ketorolac, ibuprofen, naproxen, flurbiprofen, ketoprofen, fenoprofin, oxaprozin, mefenamic acid, meclofenamic acid, piroxicam, meloxicam, nabumetone, rofecoxib, celecoxib, etodolac, nimesulide, aceclofenac, alclofenac, alminoprofen, amfenac, ampiroxicam, apazone, araprofen, azapropazone, bendazac, benoxaprofen, benzydamine, bermoprofen, benzpiperylon, bromfenac, bucloxic acid, bumadizone, butibufen, carprofen, cimicoxib, cinmetacin, cinnoxicam, clidanac, clofezone, clonixin, clopirac, darbufelone, deracoxib, droxicam, eltenac, enfenamic acid, epirizole, esflurbiprofen, ethenzamide, etofenamate, etoricoxib, felbinac, fenbufen, fenclofenac, fenclozic acid, fenclozine, fendosal, fentiazac, feprazone, filenadol, flobufen, florifenine, flosulide, flubichin methanesulfonate, flufenamic acid, flufenisal, flunixin, flunoxaprofen, fluprofen, fluproquazone, furofenac, ibufenac, imrecoxib, indoprofen, isofezolac, isoxepac, isoxicam, licofelone, lobuprofen, lomoxicam, lonazolac, loxaprofen, lumaricoxib, mabuprofen, miroprofen, mofebutazone, mofezolac, morazone, nepafanac, niflumic acid, nitrofenac, nitroflurbiprofen, nitronaproxen, orpanoxin, oxaceprol, oxindanac, oxpinac, oxyphenbutazone, pamicogrel, parcetasal, parecoxib, parsalmide, pelubiprofen, pemedolac, phenylbutazone, pirazolac, pirprofen, pranoprofen, salicin, salicylamide, salicylsalicylic acid, satigrel, sudoxicam, suprofen, talmetacin, talniflumate, tazofelone, tebufelone, tenidap, tenoxicam, tepoxalin, tiaprofenic acid, tiaramide, tilmacoxib, tinoridine, tiopinac, tioxaprofen, tolfenamic acid, triflusal, tropesin, ursolic acid, valdecoxib, ximoprofen, zaltoprofen, zidometacin, and zomepirac.

If any of these optional components, i.e., the osmolar component, the compound that enables persistence of the composition to the surface of the bladder epithelium, the antibacterial component, the antifungal compound, the vasoconstrictor, the preservative, or the anti-inflammatory agent, are present, they are typically added after a stable solution including the heparinoid, the acute-acting anesthetic, and the buffer has been prepared. The quantities of these additional optional components, if used, are chosen such that the solution of the heparinoid, the acute-acting anesthetic, and the buffer remains stable and precipitation of the acute-acting anesthetic is avoided and the final pH of the solution is achieved; the final pH is typically from about 6.8 to about 8.3 as described above. An optimum pH is about 7.3 to about 7.6, preferably about 7.5.

If sterilization of the composition is required, it is typically performed by filtration. Other sterilization methods are known in the art, including heat sterilization.

Accordingly, one aspect of the present invention is a method for preparing a composition useful for treatment of a lower urinary tract disease or condition comprising a heparinoid, an acute-acting anesthetic, and a buffer, the method comprising the steps of:

(1) providing a heparinoid, either as a solid or as an aqueous liquid, in a quantity of about 100 units to about 250,000 units per unit dose, or, alternatively, from about 4 mg to about 1000 mg per unit dose;

(2) providing an acute-acting anesthetic, either as a solid or as an aqueous liquid, in a quantity of from about 5 mg to about 1000 mg per unit dose;

(3) combining the heparinoid and the acute-acting anesthetic; and

(4) buffering the combination of the heparinoid and the acute-acting anesthetic of step (3) to a pH value of greater than about 6.8 to about 8.3 with a buffer and the possible addition of a base selected from the group consisting of sodium hydroxide and potassium hydroxide compatible with both the heparinoid and the acute-acting anesthetic to form a stable solution.

Another aspect of the present invention is a method for preparing a composition useful for treatment of a lower urinary tract disease or condition comprising a heparinoid, an acute-acting anesthetic, and a buffer, the method comprising the steps of:

(1) providing a heparinoid, either as a solid or as an aqueous liquid, in a quantity of about 100 units to about 250,000 units per unit dose, or, alternatively, from about 0.5 mg to about 1250 mg per unit dose;

(2) buffering the heparinoid to a pH value of greater than about 6.8 to about 8.3 with a buffer compatible with both the heparinoid and an acute-acting anesthetic that is to be added subsequently;

(3) adding an acute-acting anesthetic, either as a solid or as an aqueous liquid, in a quantity of from about 5 mg to about 1000 mg per unit dose, to the buffered heparinoid from step (2) to form a solution including heparinoid, acute-acting anesthetic, and buffer; and

(4) if required, rebuffering the solution of step (3) to a pH value of greater than about 6.8 to about 8.3 to form a stable solution, using buffer or sodium hydroxide.

Suitable or preferred alternatives for the heparinoid, the acute-acting anesthetic, and the buffer are described above. In one preferred alternative, the heparinoid is heparin, the acute-acting anesthetic is lidocaine, and the buffer is bicarbonate buffer, Tris buffer or phosphate buffer. In one particularly preferred alternative, the heparinoid is heparin sodium, the acute-acting anesthetic is lidocaine, and the buffer is sodium bicarbonate buffer, Tris buffer or sodium phosphate buffer.

As detailed above, the heparinoid and the acute-acting anesthetic can be provided either in solid (e.g., powdered) form or in aqueous liquid form prior to the mixing process. All possible combinations of solid form and aqueous liquid form are possible for these processes; it is possible to use: (i) both the heparinoid and the acute-acting anesthetic in solid form; (ii) both the heparinoid and the acute-acting anesthetic in aqueous liquid form; (iii) the heparinoid in solid form, with the acute-acting anesthetic in aqueous liquid form; or (iv) the heparinoid in aqueous liquid form with the acute-acting anesthetic in solid form. However, as detailed below, when the heparinoid is heparin and the acute-acting anesthetic is lidocaine, it is necessary to employ powdered heparin and powdered lidocaine hydrochloride in the alternative processes described above, because available heparin and lidocaine hydrochloride solutions are not compatible upon the addition of buffer and the lidocaine precipitates regardless of subsequent attempts to avoid precipitation and maintain the lidocaine in solution. The resulting solution containing a heparinoid stabilizes the lidocaine at least partially as a free base; typically, from about 2% to about 45% of the lidocaine is present in the free base form.

In one preferred alternative, the process comprises:

(1) mixing the heparinoid and the acute-acting anesthetic to produce a liquid form in which the heparinoid and the acute-acting anesthetic are slightly more concentrated than in the final product;

(2) adding the buffer to produce a pH of about 7.0 to 7.3 in the solution of (1); and

(3) raising the pH to a value in the range of from about 7.1 to about 8.3 using sodium hydroxide and adding water as required to achieve the final desired concentrations of the heparinoid and the acute-acting anesthetic.

Another aspect of the present invention is a stable composition comprising a heparinoid, an acute-acting anesthetic, and a buffer. The stable composition can be prepared by the process described above. Typically, in this composition, the stability of the heparinoid and the acute-acting anesthetic is at least 90% after one year, up to 18 months. Preferably, in this composition, the stability of the heparinoid and the acute-acting anesthetic is at least 95% after one year, More preferably, in this composition, the stability of the heparinoid and the acute-acting anesthetic is at least 97% after one year up to 18 months. As used herein, the terms “95% stability” or “97% stability” in reference to either the heparinoid or the acute-acting anesthetic are defined as meaning that 95% or 97% of the original concentration of the heparinoid or the acute-acting anesthetic remains in the composition in its original physical state and is bioavailable; heparinoid or acute-acting anesthetic that has precipitated or decomposed is excluded by this definition. The stability is determined from the time when the final product or vial containing it is prepared, so that any prior loss during purification, filtration, or autoclaving is not taken into account in determining the percentage of stability. Typically, when the acute-acting anesthetic is lidocaine, from about 2% to about 45% of the lidocaine is present in the free base form.

Suitable heparinoids, acute-acting anesthetics, and buffers for compositions according to the present invention are as described above. Suitable quantities of the heparinoid, the acute-acting anesthetic, and the buffer per unit dose for compositions according to the present invention are as described above.

The pH value of the composition is in the range of greater than about 6.8 to about 8.3. Preferably, the pH value of the composition is from about 7.2 to about 7.6. More preferably, the pH value of the composition is about 7.3 to 7.5.

A preferred composition according to the present invention comprises heparin as the heparinoid, lidocaine as the acute-acting anesthetic, and bicarbonate buffer as the buffer. A particularly preferred composition according to the present invention comprises heparin sodium as the heparinoid, lidocaine hydrochloride as the acute-acting anesthetic, and sodium bicarbonate, Tris or sodium phosphate as the buffer.

Compositions according to the present invention can further comprise one or more additional optional components as described above. Such additional optional components can include:

(1) an osmolar component that provides an isotonic or nearly isotonic solution compatible with human cells and blood;

(2) a compound that enables persistence of the composition to the surface of the bladder epithelium in a quantity sufficient to treat, ameliorate, or prevent a lower urinary tract disorder;

(3) an antibacterial agent in a quantity sufficient to treat, ameliorate, or prevent a lower urinary tract disorder;

(4) an antifungal agent in a quantity sufficient to treat, ameliorate, or prevent a lower urinary tract disorder;

(5) a vasoconstrictor in a quantity sufficient to treat, ameliorate, or prevent a lower urinary tract disorder;

(6) a preservative; and

(7) an anti-inflammatory agent.

Compositions according to the present invention can be formulated for or are suitable for treating, ameliorating, or preventing a lower urinary tract disorder selected from the group consisting of bacterial cystitis, fungal/yeast cystitis, vulvar vestibulitis, vulvodynia, dyspareunia, urethral syndrome, and endometriosis in women; prostatitis and chronic pelvic pain syndrome in men; and radiation-induced cystitis, chemotherapy-induced cystitis, interstitial cystitis, and overactive bladder in men or women. Compositions according to the present invention are particularly useful in treating interstitial cystitis.

As used herein, the terms “treat, ameliorate, or prevent” refer to any detectable improvement, whether subjective or objective, in the lower urinary tract disorder of the subject to whom the composition is administered. For example, the terms “treat, ameliorate, or prevent” can refer to an improvement as determined by the PORIS scale, the PUF scale, or any component of those scales; reduction of pain; reduction of urinary frequency; reduction of urinary urgency; reduction of requirement for narcotic administration; reduction of incontinence; reduction of abnormal permeability of the urothelium to potassium; or improvement in more than one of these parameters. The terms “treat, ameliorate, or prevent” do not state or imply a cure for the underlying lower urinary tract disorder.

Accordingly, yet another aspect of the invention is a method for treating, ameliorating, or preventing a lower urinary tract disorder comprising instillation of a therapeutically effective quantity of a composition according to the present invention into the bladder of a subject in need thereof, wherein the lower urinary tract disorder is selected from the group consisting of bacterial cystitis, fungal/yeast cystitis, vulvar vestibulitis, vulvodynia, dyspareunia, urethral syndrome, and endometriosis in women; prostatitis and chronic pelvic pain syndrome in men; and radiation-induced cystitis, chemotherapy-induced cystitis, interstitial cystitis, and overactive bladder in men or women. A particularly significant lower urinary tract disorder suitable for treatment by use of a composition according to the present invention is interstitial cystitis.

Methods of instillation of compositions comprising a heparinoid, an acute-acting anesthetic, and a buffer are described, for example, in U.S. Pat. No. 7,414,039 by Parsons, incorporated herein by this reference.

The invention is illustrated by the following Examples. These Examples are included for illustrative purposes only, and are not intended to limit the invention.

### Example 1

### Composition with Heparin, Lidocaine, and Bicarbonate

Heparin (50,000 units or 250 mg plus lidocaine (200 mg) was buffered with sodium bicarbonate to a pH of 7.5 and in a final volume of 15 mL. After both 12 and 18 months, the heparin and lidocaine were both over 95% stable.

### Example 2

### Composition with Heparin, Lidocaine, and Phosphate (Prospective Example)

Heparin (50,000 units or 250 mg) plus lidocaine (200 mg) is buffered with phosphate to a pH of 7.5 and in a final volume of 15 mL. After both 12 and 18 months, the heparin and lidocaine were expected to be both over 95% stable.

### Example 3

### Stability and Absorption for Composition with Heparin, Lidocaine, and Phosphate

A clinical trial to evaluate the stability and absorption of lidocaine for a composition with heparin, lidocaine, and phosphate versus lidocaine alone was undertaken. For the serum lidocaine level study the heparin and lidocaine solution was 25 mL containing 333 mg lidocaine hydrochloride and 50,000 units of heparin buffered to a pH of about 7.1-7.2 with phosphate buffer and was obtained from a specialty compounding pharmacy. For lidocaine, a 25-mL solution was prepared using lidocaine hydrochloride, containing 333 mg lidocaine hydrochloride, with a pH of about 6.3 (no buffer added). These products were instilled into the urinary bladders of interstitial cystitis patients and after 45 minutes blood was drawn to measure the serum lidocaine levels. The lidocaine levels were determined by HPLC.

Results of serum lidocaine in patients receiving heparin-lidocaine compared to lidocaine only are shown in Table 1. Lidocaine was significantly better absorbed (2.25 fold) when both drugs are used. The conclusions were that the heparin helped stabilize the lidocaine and prevent its precipitation and this resulted in a more than 2-fold increase in lidocaine serum levels. Also, the lidocaine only solution was unstable with precipitation of lidocaine.

TABLE 1

| Group | N | serum lidocaine level | p value* |
|---|---|---|---|
| Heparin plus | 11 | 0.45 ug/Ml | 0.019 |
| Lidocaine (Buffered) | | | |
| Lidocaine | 10 | 0.20 ug/Ml | |

In Table 1, the p value was calculated using Student's t test. As can be seen, the heparin and lidocaine solution resulted in significantly better absorption of lidocaine compared to the lidocaine only solution.

The data show clearly that the product with heparin, lidocaine, and phosphate buffer results in better absorption compared to lidocaine alone. It also shows that the product did not precipitate when the solution was alkalinized compared to lidocaine alone. The data also support the concept of the inventor that the heparin helps stabilize the lidocaine, the result being more than twice the absorption of lidocaine into the bladder wall.

### ADVANTAGES OF THE INVENTION

The present invention provides improved compositions for treatment of a lower urinary tract disorder that include a heparinoid, an acute-acting anesthetic, and a buffer. Typically, the heparinoid is heparin and the acute-acting anesthetic is lidocaine. Typically, the buffer is phosphate buffer, Tris buffer, or sodium bicarbonate. Compositions prepared by the method of the present invention are stable for twelve months or more and do not undergo precipitation of the acute-acting anesthetic. Accordingly, they retain efficacy and the acute-acting anesthetic retains bioavailability, an improvement over previously available compositions. A major advantage of these compositions is the use of a highly sulfonated GAG compound to stabilize lidocaine in soluble form in a solution at higher pH causing alkalization of the lidocaine to its active free base. Moreover, it has unexpectedly been shown that heparinoids both stabilize the acute-acting anesthetic, such as lidocaine, in the composition, and promote absorption of the acute-acting anesthetic, such as lidocaine, by the urothelium.

Compositions according to the present invention possess industrial applicability as compositions intended for medical use, specifically to treat lower urinary tract diseases and conditions. Methods according to the present invention possess industrial applicability for the preparation of a medicament to treat lower urinary tract diseases and conditions.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, and literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

1. A method for preparing a composition useful for treatment of a lower urinary tract disease or condition comprising a heparinoid, an acute-acting anesthetic, and a buffer, the method comprising the steps of:
(a) providing a heparinoid, either as a solid or as an aqueous liquid, in a quantity of about 100 units to about 250,000 units per unit dose, or, alternatively, from about 0.5 mg to about 1250 mg per unit dose;
(b) providing an acute-acting anesthetic, either as a solid or as an aqueous liquid, in a quantity of from about 5 mg to about 1000 mg per unit dose;
(c) combining the heparinoid and the acute-acting anesthetic; and
(d) buffering the combination of the heparinoid and the acute-acting anesthetic of step (c) to a pH value of greater than about 6.8 to about 8.3 with a buffer and the possible addition of a base selected from the group consisting of sodium hydroxide and potassium hydroxide compatible with both the heparinoid and the acute-acting anesthetic to form a stable solution, wherein the stability of the heparinoid and the acute-acting anesthetic in the stable solution is at least 90% after one year, up to 18 months.

2. The method of claim 1 wherein the pH value is from about 7.2 to about 7.6.

3. (canceled)

4. The method of claim 1 wherein the heparinoid is selected from the group consisting of heparin, chondroitin sulfate, heparan sulfate, hyaluronic acid, keratan sulfate, dermatan sulfate, hyaluronan, and sodium pentosanpolysulfate.

5. The method of claim 4 wherein the heparinoid is selected from the group consisting of heparin and sodium pentosanpolysulfate.

6.-10. (canceled)

11. The method of claim 1 wherein the acute-acting anesthetic is selected from the group consisting of benzocaine, lidocaine, tetracaine, bupivacaine, cocaine, etidocaine, flecainide, mepivacaine, pramoxine, prilocaine, procaine, chloroprocaine, oxyprocaine, proparacaine, ropivacaine, dyclonine, dibucaine, propoxycaine, chloroxylenol, dexivacaine, diamocaine, hexylcaine, levobupivacaine, propoxycaine, pyrrocaine, risocaine, rodocaine, and pharmaceutically acceptable derivatives and bioisosteres thereof, and a combination thereof.

12. (canceled)

13. The method of claim 11 wherein the acute-acting anesthetic is lidocaine.

14. (canceled)

15. The method of claim 1 wherein the buffer is selected from the group consisting of phosphate buffer, bicarbonate buffer, Tris (Tris(hydroxymethyl)aminomethane) buffer, MOPS buffer (3-(N-morpholino)propanesulfonic acid), HEPES (N-(2-hydroxyethyl)piperazine-N-(2-ethanesulfonic acid) buffer, ACES (2-[(2-amino-2-oxoethyl)amino]ethanesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid) buffer, HEPPSO (N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer, TAPS (N-tris[hydroxymethyl)methyl-3-aminopropanesulfonic acid) buffer; TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, 2-amino-2-methyl-1-propanol buffer, and a combination thereof.

16. The method of claim 15 wherein the buffer is selected from the group consisting of bicarbonate buffer, phosphate buffer, Tris buffer, and a combination thereof.

17.-20. (canceled)

21. The method of claim 1 wherein:
(a) both the heparinoid and the acute-acting anesthetic are provided in solid form;
(b) both the heparinoid and the acute-acting anesthetic are provided in liquid form;
(c) the heparinoid is provided in solid form and the acute-acting anesthetic is provided in liquid form; or
(d) the heparinoid is provided in liquid form and the acute-acting anesthetic is provided in solid form.

22.-27. (canceled)

28. The method of claim 1 wherein the heparinoid is heparin, the acute-acting anesthetic is lidocaine, and the buffer is bicarbonate buffer or phosphate buffer.

29. (canceled)

30. The method of claim 5 wherein the heparinoid is heparin and wherein the quantity of heparin in the composition is from about 1000 units to about 250,000 units per unit dose of the composition, or, alternatively, from about 0.5 mg to about 1250 mg per unit dose of the composition.

31.-37. (canceled)

38. A method for preparing a composition useful for treatment of a lower urinary tract disease or condition comprising a heparinoid, an acute-acting anesthetic, and a buffer, the method comprising the steps of:
(a) providing a heparinoid, either as a solid or as an aqueous liquid, in a quantity of about 100 units to about 250,000 units per unit dose or, alternatively, from about 0.5 mg to about 1250 mg per unit dose;
(b) buffering the heparinoid to a pH value of greater than about 6.8 to about 8.3 with a buffer compatible with both the heparinoid and an acute-acting anesthetic that is to be added subsequently;
(c) adding an acute-acting anesthetic, either as a solid or as an aqueous liquid, in a quantity of from about 5 mg to about 1000 mg per unit dose, to the buffered heparinoid from step (b) to form a solution including heparinoid, acute-acting anesthetic, and buffer; and
(d) if required, rebuffering the solution of step (c) to a pH value of greater than about 6.8 to about 8.3 to form a stable solution, wherein the stability of the heparinoid and the acute-acting anesthetic is at least 90% after one year, up to 18 months.

39-40. (canceled)

41. The method of claim 38 wherein the heparinoid is selected from the group consisting of heparin, chondroitin sulfate, heparan sulfate, hyaluronic acid, keratan sulfate, dermatan sulfate, hyaluronan, and sodium pentosanpolysulfate.

42. The method of claim 41 wherein the heparinoid is selected from the group consisting of heparin and sodium pentosanpolysulfate.

43.-47. (canceled)

48. The method of claim 38 wherein the acute-acting anesthetic is selected from the group consisting of benzocaine, lidocaine, tetracaine, bupivacaine, cocaine, etidocaine, flecainide, mepivacaine, pramoxine, prilocaine, procaine, chloroprocaine, oxyprocaine, proparacaine, ropivacaine, dyclonine, dibucaine, propoxycaine, chloroxylenol, dexivacaine, diamocaine, hexylcaine, levobupivacaine, propoxycaine, pyrrocaine, risocaine, rodocaine, and pharmaceutically acceptable derivatives and bioisosteres thereof, and a combination thereof.

49. (canceled)

50. The method of claim 48 wherein the acute-acting anesthetic is lidocaine.

51. (canceled)

52. The method of claim 38 wherein the buffer is selected from the group consisting of bicarbonate buffer, phosphate buffer, Tris (Tris(hydroxymethyl)aminomethane) buffer, MOPS buffer (3-(N-morpholino)propanesulfonic acid), HEPES (N-(2-hydroxyethyl)piperazine-N-(2-ethanesulfonic acid) buffer, ACES (2-[(2-amino-2-oxoethyl)amino]ethanesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid) buffer, HEPPSO (N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer, TAPS (N-tris[hydroxymethyl)methyl-3-aminopropanesulfonic acid) buffer; TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, 2-amino-2-methyl-1-propanol buffer, and a combination thereof.

53. The method of claim 52 wherein the buffer is selected from the group consisting of bicarbonate buffer, phosphate buffer, Tris buffer, and a combination thereof.

54.-57. (canceled)

58. The method of claim 38 wherein:
(a) both of the heparinoid and the acute-acting anesthetic are provided in solid form;
(b) both of the heparinoid and the acute-acting anesthetic are provided in liquid form;
(c) the heparinoid is provided in liquid form and the acute-acting anesthetic is provided in solid form; or
(d) the heparinoid is provided in solid form and the acute-acting anesthetic is provided in liquid form.

59.-64. (canceled)

65. The method of claim 38 wherein the heparinoid is heparin, the acute-acting anesthetic is lidocaine, and the buffer is bicarbonate buffer, Tris buffer, or phosphate buffer.

66.-67. (canceled)

68. The method of claim 43 wherein the quantity of heparin in the composition is from about 1000 units to about 250,000 units per unit dose of the composition or, alternatively, from about 0.5 mg to about 1250 mg per unit dose of the composition.

69.-75. (canceled)

76. A method for preparing a composition useful for treatment of a lower urinary tract disease or condition comprising a heparinoid, an acute-acting anesthetic, and a buffer, the method comprising the steps of:
(a) mixing the heparinoid and the acute-acting anesthetic to produce a liquid form in which the heparinoid and the acute-acting anesthetic are slightly more concentrated than in the final product;
(b) adding the buffer to produce a pH of about 7.0 in the solution of (a); and
(c) raising the pH to a value in the range of from about 7.1 to about 8.3 using sodium hydroxide and adding water as required to achieve the final desired concentrations of the heparinoid and the acute-acting anesthetic, wherein the stability of the heparinoid and the acute-acting anesthetic is at least 90% after one year, up to 18 months.

77. The method of claim 76 wherein the pH achieved in step (c) is about 7.5.

78. The method of claim 76 wherein the heparinoid is selected from the group consisting of heparin, chondroitin sulfate, heparan sulfate, hyaluronic acid, keratan sulfate, dermatan sulfate, hyaluronan, and sodium pentosanpolysulfate.

79. The method of claim 78 wherein the heparinoid is selected from the group consisting of heparin and sodium pentosanpolysulfate.

80.-84. (canceled)

85. The method of claim 76 wherein the acute-acting anesthetic is selected from the group consisting of benzocaine, lidocaine, tetracaine, bupivacaine, cocaine, etidocaine, flecainide, mepivacaine, pramoxine, prilocaine, procaine, chloroprocaine, oxyprocaine, proparacaine, ropivacaine, dyclonine, dibucaine, propoxycaine, chloroxylenol, dexivacaine, diamocaine, hexylcaine, levobupivacaine, propoxycaine, pyrrocaine, risocaine, rodocaine, and pharmaceutically acceptable derivatives and bioisosteres thereof, and a combination thereof.

86. (canceled)

87. The method of claim 85 wherein the acute-acting anesthetic is lidocaine.

88. (canceled)

89. The method of claim 76 wherein the buffer is selected from the group consisting of bicarbonate buffer, phosphate buffer, Tris (Tris(hydroxymethyl)aminomethane) buffer, MOPS buffer (3-(N-morpholino)propanesulfonic acid), HEPES (N-(2-hydroxyethyl)piperazine-N-(2-ethanesulfonic acid) buffer, ACES (2-[(2-amino-2-oxoethyl)amino]ethanesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid) buffer, HEPPSO (N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer, TAPS (N-tris[hydroxymethyl)methyl-3-aminopropanesulfonic acid) buffer; TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, 2-amino-2-methyl-1-propanol buffer, and a combination thereof.

90. The method of claim 89 wherein the buffer is selected from the group consisting of bicarbonate buffer, phosphate buffer, Tris buffer, and a combination thereof.

91-95. (canceled)

96. The method of claim 76 wherein the heparinoid is heparin, the acute-acting anesthetic is lidocaine, and the buffer is bicarbonate buffer, phosphate buffer, or Tris buffer.

97.-98. (canceled)

99. The method of claim 80 wherein the quantity of heparin in the composition is from about 1000 units to about 250,000 units per unit dose of the composition or, alternatively, from about 0.5 mg to about 1250 mg per unit dose.

100.-106. (canceled)

107. A stable composition comprising a heparinoid, an acute-acting anesthetic, and a buffer wherein the stability of the heparinoid and the acute-acting anesthetic is at least 90% after one year, up to 18 months.

108.-109. (canceled)

110. The composition of claim 107 wherein the stability of the heparinoid and the acute-acting anesthetic is at least 97% after one year, up to 18 months.

111. (canceled)

112. The composition of claim 107 wherein the composition is prepared by a process comprising the steps of:
(a) providing a heparinoid, either as a solid or as an aqueous liquid, in a quantity of about 100 units to about 250,000 units per unit dose, or, alternatively, in a quantity of from about 0.5 mg to about 1250 mg per unit dose;
(b) providing an acute-acting anesthetic, either as a solid or as an aqueous liquid, in a quantity of from about 5 mg to about 1000 mg per unit dose;
(c) combining the heparinoid and the acute-acting anesthetic; and
(d) buffering the combination of the heparinoid and the acute-acting anesthetic of step (c) to a pH value of greater than about 6.8 to about 8.3 with a buffer compatible with both the heparinoid and the acute-acting anesthetic to form a stable solution.

113. The composition of claim 107 wherein the composition is prepared by a process comprising the steps of:
(a) providing a heparinoid, either as a solid or as an aqueous liquid, in a quantity of about 100 units to about 250,000 units per unit dose, or, alternatively, in a quantity of from about 0.5 mg to about 1250 mg per unit dose;
(b) buffering the heparinoid to a pH value of greater than about 6.8 to about 8.3 with a buffer compatible with both the heparinoid and an acute-acting anesthetic that is to be added subsequently;
(c) adding an acute-acting anesthetic, either as a solid or as an aqueous liquid, in a quantity of from about 5 mg to about 1000 mg per unit dose, to the buffered heparinoid from step (2) to form a solution including heparinoid, acute-acting anesthetic, and buffer; and
(d) if required, rebuffering the solution of step (c) to a pH value of greater than about 6.8 to about 8.3 to form a stable solution.

114. The composition of claim 107 wherein the composition is prepared by a process comprising the steps of:
(a) mixing the heparinoid and the acute-acting anesthetic to produce a liquid form in which the heparinoid and the acute-acting anesthetic are slightly more concentrated than in the final product;
(b) adding the buffer to produce a pH of about 7.0 in the solution of (a); and
(c) raising the pH to a value in the range of from about 7.1 to about 8.3 using sodium hydroxide and adding water as required to achieve the final desired concentrations of the heparinoid and the acute-acting anesthetic.

115.-143. (canceled)

144. The composition of claim 107 wherein the composition is formulated for treating, ameliorating, or preventing a lower urinary tract disorder selected from the group consisting of bacterial cystitis, fungal/yeast cystitis, vulvar vestibulitis, vulvodynia, dyspareunia, urethral syndrome, and endometriosis in women; prostatitis and chronic pelvic pain syndrome in men; and radiation-induced cystitis, chemotherapy-induced cystitis, interstitial cystitis, and overactive bladder in men or women.

145. The composition of claim 144 wherein the composition is formulated for treating, ameliorating, or preventing interstitial cystitis.

146.-147. (canceled)
